(19) United States
(12) Patent Application Publication
(10) Pub. No.: US 2025/0186998 A1
(43) Pub. Date: Jun. 12, 2025

(54) Cartridge for Point-Of-Care Assessment of Biological Analytes and a System And Method Thereof

(71) Applicant: Zulma Guillot, Surfside, FL (US)

(72) Inventors: Zulma Guillot, Surfside, FL (US); Dinesh Kumar, New Delhi (IN)

(73) Assignee: Zulma Guillot, Surfside, FL (US)

(21) Appl. No.: 18/805,421

(22) Filed: Aug. 14, 2024

Related U.S. Application Data
(60) Provisional application No. 63/532,905, filed on Aug. 15, 2023.

(51) Int. Cl. B01L 3/00

(52) U.S. Cl. CPC B01L 3/502715; B01L 2200/0647; B01L 2300/0816

(57) ABSTRACT

The present invention relates generally to a system and method for detecting the plurality of analytes in a blood sample. The present invention involves an advanced cartridge designed for detecting multiple analytes in a blood sample. The cartridge integrates a series of components to process the blood sample, beginning with its reception and separation into plasma. The plasma is then precisely measured and directed through an incubation process, where it is mixed with reagents and incubated under controlled conditions. Following incubation, the plasma is analyzed using lateral flow strips that detect and quantify various target analytes. This cartridge system is particularly suited for point-of-care testing, providing a streamlined, automated approach to performing complex assays, including immunoassays and electrochemical assays, with enhanced accuracy and efficiency.

### CROSS-REFERENCE TO RELATED APPLICATION

This disclosure claims the benefit of the priority of U.S. Provisional Patent Application No. 63/532,905 entitled “Point Of Care System For Assessment Of Biological Analytes” and filed on Aug. 15, 2023. The above-identified application is incorporated herein by reference in its entirety.

### TECHNICAL FIELD

The invention relates to a point-of-care (POC) device and, more particularly, to a cartridge for the assessment of biological analytes at point-of-care; furthermore, to a system and its method for the quantitative evaluation of multiple biological analytes in a blood sample point-of-care. Specifically, the invention of a point-of-care device that works in conjunction with a multiplex cartridge provides a quantitative assessment of biological analytes at the point of care.

### BACKGROUND

The background description includes information that may be useful in understanding the present invention. It is not an admission that any of the information provided herein is prior art or relevant to the presently claimed invention, or that any publication specifically or implicitly referenced is prior art.

Point-of-care (POC) testing has become increasingly important for providing rapid and accurate diagnostic results directly at the site of the patient care. Traditional testing methods, such as those involving lipid panels or hormonal assessments, often require patients to visit a laboratory where blood samples are collected and then sent off site for analysis. This process can be time-consuming, with results sometimes taking several days to return. Consequently, there is a pressing need for on-site testing systems that offer swift results and the capability to assess multiple analytes from a single patient sample. More particularly, there is urgent need for a cartridge for on-site testing of important female hormones including estradiol, progesterone, testosterone, TSH, and FSH simultaneously in a single cartridge.

In the realm of hormonal assays, POC systems are expected to measure various hormones such as estradiol, progesterone, testosterone, thyroid-stimulating hormone (TSH), and FSH with high sensitivity and accuracy. Moreover, there is a current need to measure estradiol, progesterone, testosterone, TSH and FSH in female to assess various important factors in females. Established benchmarks, such as those provided by the Mayo Clinic, set a high standard for sensitivity and reference ranges. These standards are designed to cover 90 to 95% of the general population within each clinical subgroup, ensuring that tests can accurately determine hormonal levels for the majority of patients.

However, achieving these high standards in a POC setting presents several challenges. Traditional approaches to hormonal assays often involve automated liquid pipetting stations that use enzyme-linked immunosorbent assay (ELISA) techniques. These systems benefit from advanced electrochemical detection, effective washing procedures, efficient mixing, and precise pipetting mechanisms, allowing them to deliver high sensitivity and a wide analytical range. Despite their effectiveness, these systems are typically large, complex, and unsuitable for rapid POC use due to their need for extensive laboratory infrastructure.

Current POC devices often use fluorescence-based lateral flow assays for hormonal testing. These systems, manufactured by companies like Boditech, Getein, VEDA Labs, and Getein Biotech Inc., use standard single-plex lateral flow strips housed in rectangular casings. Some of these devices feature dedicated fluorescence readers and incubators to maintain the correct temperature during testing. Although these systems can perform some hormone assays, they generally require plasma or serum rather than finger stick blood, which limits their convenience. Additionally, their performance often falls short of the “Gold Standard” sensitivity ranges established by research-style methods. For example, none of these systems offer lateral flow estradiol assay, and their coefficient of variation (CV) values are higher. CK values are <20% at least Bodytech and CTK Biotech indicating less reliable results.

Although advancements in miniaturized medical systems and the discovery of numerous disease biomarkers have paved the way for improved POC testing, significant challenges remain. Current devices often struggle to achieve the high sensitivity and versatility required for comprehensive hormonal assays and other tests. Innovations that address these limitations such as improving reagent stability, and enhancing the number of assays that can be performed in parallel, are essential for the development of more effective and accessible POC diagnostic tools.

### SUMMARY

The present invention relates to a point of care (POC) blood analyser and a cartridge and a system that addresses the limitations of the traditional diagnostic methods by providing rapid, accurate results directly to the physician. The system also overcomes the challenges faced by existing devices, including the need for extensive lab infrastructure and limitations in sensitivity and convenience. By incorporating advanced technologies and innovative design, it ensures high reliability, and supports a wider range of hormonal analytes, making it a significant advancement in POC diagnostics. The invention also provides an efficient method of assessment of various analytes in a blood sample using a cartridge discloses herein.

In an embodiment of the present invention, the invention discloses an automated cartridge for detection of a plurality of analytes in a blood sample of a person. In the embodiment, the plurality of analytes are five hormones includes estradiol, progesterone, testosterone, TSH and FSH in females. The cartridge comprises a blood tube receiver to receive the blood sample of the patient into the cartridge, a plasma separator positioned between a blood collection chamber and a plasma chamber to filter the plasma from the blood sample into the plasma chamber. In addition, the cartridge includes a plasma metering chamber configured to receive the plasma and a pumping mechanism to pump the plasma to flow from the plasma metering chamber to an overflow chamber. Further, the overflow chamber diverts the plasma to a plurality of incubation wells that comprises a plurality of magnetic beads and chemical reagents. Furthermore, a plurality of lateral flow strips is configured to receive the incubated plasma from the plurality of incubation wells after a trehalose plug is broken due to magnetic agitation under a magnetic field.

In one of the embodiments of the present invention, the pumping mechanism comprises a pump with an aspirate plunger having cantilever snaps or a pump with double stopper.

In one of the embodiments of the present invention, the pumping mechanism comprises a valve to prevent backflow of the sample.

In one of the embodiments of the present invention, a thermally conductive printed circuit board comprises a plurality of magnetic coil, a plurality of printed heater, a temperature sensor and a power input source. The thermally conductive printed circuit is configured to generate a revolving magnetic field in each of the plurality of magnetic coil.

In one of the embodiments of the present invention, the plurality of magnetic beads is moved under a magnetic field induced by a thermally conductive printed circuit board that is placed underneath the plurality of the incubation wells. The agitation caused due to the magnetic field allows the magnetic bead to move and mix the blood samples with the chemical reagents effectively.

In one of the embodiments of the present invention, the plasma is incubated for 10-30 min at 37 degree Celsius in the plurality of incubation wells. In one of the embodiments, the plasma is incubated for less than 10 min at 37 degree Celsius in the plurality of incubation wells. In one of the embodiments of the present invention, the trehalose plug is made up of cellulose.

In one of the embodiments of the present invention, the plurality of lateral flow strips comprises a control line and a test line to detect the presence of the plurality of analytes.

In one of the embodiments of the present invention, each of the plurality of incubation wells is surrounded by a conductive plastic.

In the embodiment of the present invention, the cartridge is made up of thermoplastic material.

In one of the embodiments of the present invention, the plurality of magnetic beads is made up of ceramic and have measuring 1.5 mm×4 mm.

In an embodiment of the present invention, the invention discloses a computer implemented system for detection of a plurality of analytes in a blood sample. The system comprises a measuring instrument, a cartridge and a data system. The measuring instrument includes a cartridge id logging module for storing the person credentials, a temperature control module to maintain temperature conditions for an effective incubation, a touchscreen interface module for displaying the results of a tested blood sample of the person, a diagnostic module for scheduling system maintenance, a signal processing module for calculating results by analysing a fluorescence signal for a plurality of lateral flow strips within the inserted cartridge and a imaging module that is integrated with an optical module for processing image data, detecting test lines, reading results, and correcting artifacts. The optical module is used for precise capturing of the test results on the lateral flow strips. In addition, the instrument includes a sensor module to ensure proper functioning of the instrument modules, a power source module for providing electrical energy to power the instrument components and a cartridge receiver to facilitate the entry of the cartridge into the system. In one of the embodiments, battery may be used as power source which may be a rechargeable battery. Further, the computer implemented system comprises the cartridge that includes a blood tube receiver, a plasma separator, a blood collection chamber, a plasma chamber, a plasma metering chamber, a pumping mechanism, an overflow chamber, a plurality of incubation wells and a plurality of lateral flow strips. Furthermore, the system includes a data system to store the instrument data and measurements results that includes the quantity of analytes present in the person's blood sample.

In one the embodiment of the present invention, the data system is a remote system that stores data virtually on cloud.

In an embodiment of the present invention, the system discloses a cartridge coupled with a thermally conductive printed circuit board. The thermally conductive printed circuit board is having a plurality of printed heater, a plurality of magnetic coil and a power source input. The system depicts the thermally conductive printed circuit board receiving power from a power source input which is transferred to the plurality of printed heater and to the plurality of magnetic coils. The magnetic coils then create a magnetic field that helps in rotating the magnetic beads present inside a plurality of incubation wells. As the magnetic beads are agitated, they mix the chemical reagents with the plasma samples effectively. This movement enhances the interaction between the reagents and samples, thus leading to better mixing and potentially more accurate results in the testing process.

In an embodiment of the present invention, the invention discloses a thermally conductive printed circuit board (PCB) comprising a power source input for providing electricity to the PCB, a plurality of printed heater for regulating the temperature within the PCB and a plurality of the magnetic coil for creating a magnetic field to rotate the magnetic beads. In one of the embodiments, the magnetic field in magnetic coil may rotate in each individual coil in anticlockwise or clockwise direction.

The present disclosure further provides a fully automated method of detecting a plurality of analytes in a blood sample, the method comprising steps of inserting a collection tube into a cartridge and waiting plasma separation from the blood sample, pressing a Plunger to divert the plasma to an overflow chamber, diverting the plasma to each of a plurality of incubation wells through a plurality of interconnected channels, mixing the plasma with reagents by means of magnetic agitation induced by magnetic field of a PCB underneath a plurality of incubation wells for 10 min to 30 min, maintaining temperature of the plurality of incubation wells at 37 degrees Celsius by at least a printed heating coil, breaking a cellulose closure of a trehalose plug of the plurality of incubation wells due to magnetic agitation induced by a revolving magnetic field and diverting incubated plasma to each of a plurality of lateral flow strips, detecting results at a test site of each of a plurality of lateral flow strips; and finding results of the plurality of analytes. In the embodiment, the plurality of analytes is estradiol, progesterone, testosterone, TSH and FSH or other analytes.

In one of the embodiment of the present invention, a cartridge includes a blood tube receiver, which is angled to accept a blood tube and collects the blood sample as it flows in by gravity. The blood is then directed to a blood collection chamber. A plasma separator includes a membrane that is used for filtering the plasma component from the blood sample.

In one of the embodiment of the cartridge, the pumping mechanism includes a pump having an aspirate plunger equipped with cantilever snaps for securely locking the plunger. The pumping mechanism includes a one-way valve that is designed to permit fluid flow in only one direction, effectively blocking any reverse movement that could potentially disrupt the operation or damage the system. Additionally in this embodiment, a double stopper is used to manage the release of the plasma. In one of the embodiment, the inclusion of a ferrous mixing element or magnetic beads enhances this integration by promoting efficient blending of the plasma and reagents, ensuring that the mixing process is thorough and uniform. In addition, the mechanism features a foil layer with an air channel that regulates air pressure and manages the movement of gases within the system. The plasma is then transferred from the overflow chamber to a plurality of incubation wells via overflow channel. After the incubation process, the incubated samples is transferred to the plurality of lateral flow trips for detecting and analysing the concentration of hormones present in the person blood sample.

For further clarification of the features and other embodiments of the invention, a more particular description is provided that will further explain the features and advantage of the invention with the illustration or the drawings. As will be appreciated, other embodiments of the present invention are possible utilizing, alone or in combination, one or more of the features set forth above or described in detail below.

### BRIEF DESCRIPTION OF THE DRAWINGS

A full understanding of the invention can be gained from the following description of the preferred embodiments when read in conjunction with the accompanying drawings in which:

FIG. 1A illustrates a perspective view of a cartridge, in according to embodiments of the present invention disclosed herein;

FIG. 1B illustrates a perspective of a cartridge with a cover, in according to embodiments of the present invention disclosed herein;

FIG. 2 illustrates a perspective view of a cartridge showing internal channels, in according to embodiments of the present invention disclosed herein;

FIG. 3 illustrates a perspective view of a cartridge without a cover, in according to embodiments of the present invention disclosed herein;

FIG. 4A-FIG. 4B illustrates process flow of the blood sample in the cartridge, in according to embodiments of the present invention disclosed herein;

FIG. 5A illustrates a perspective view of a cartridge coupled with a thermally conductive printed circuit board, in according to embodiments of the present invention disclosed herein;

FIG. 5B illustrates an overview of all the components of a thermally conductive printed circuit board, in according to embodiments of the present invention disclosed herein;

FIG. 6 illustrates a perspective view of a cartridge and a measuring instrument, in according to embodiments of the present invention disclosed herein;

FIG. 7 illustrates a block diagram of a system for assessment of biological analytes, in according to embodiments of the present invention disclosed herein;

FIG. 8 illustrates various steps of a method for assessment of biological analytes, in according to embodiments of the present invention disclosed herein.

Common reference numerals are used throughout the figures and the detailed description to indicate like elements. One skilled in the art will readily recognize that the above figures are examples and that other architectures, modes of operation, orders of operation, and elements/functions can be provided and implemented without departing from the characteristics and features of the invention, as set forth in the claims.

### DETAILED DESCRIPTION

References will now be made in detail to the presently preferred embodiments of the invention, examples of which are illustrated in the accompanying drawings. Throughout the following detailed description, the same reference numerals refer to the same elements in all figures.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

The terminology used herein is for the purpose of describing particular embodiments only and it is not intended to be limiting the invention. As used herein, the term “and/or” includes any combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

In the following description, reference will be made to the accompanying drawing, in which comparable functional elements are designated with like numerals. The aforementioned accompanying drawings show by way of illustration and not by the way of limitation, specific aspects and implementations consistent with principles of this disclosure. These implementations are described in sufficient detail to enable those skilled in the art to practice the disclosure and it is to be understood that other implementations may be utilized, and that structural changes and/or substitutions of various elements may be made without departing from the scope and spirit of this disclosure. The following detailed description is, therefore, not to be construed in limited sense. It is noted that description herein is not intended as an extensive overview, and as such, concepts may be simplified in the interests of clarity and brevity. All documents mentioned in this application are hereby incorporated by reference in their entirety.

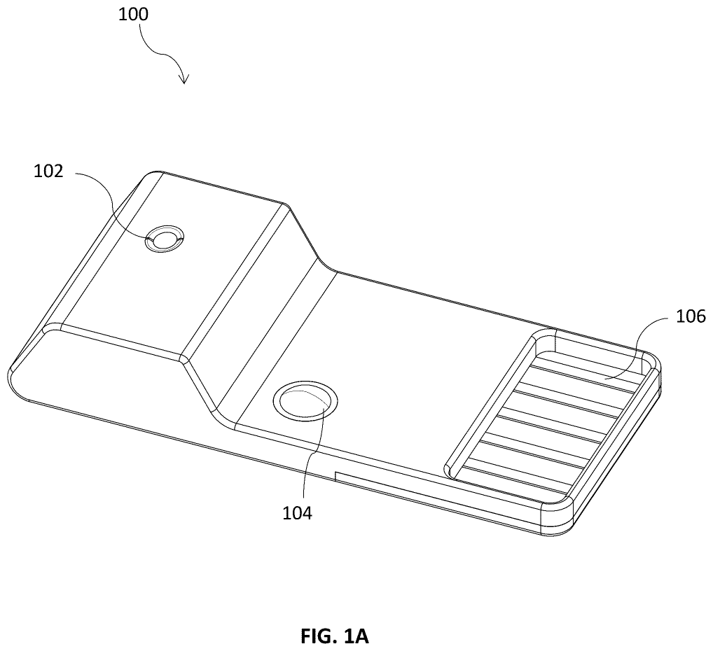

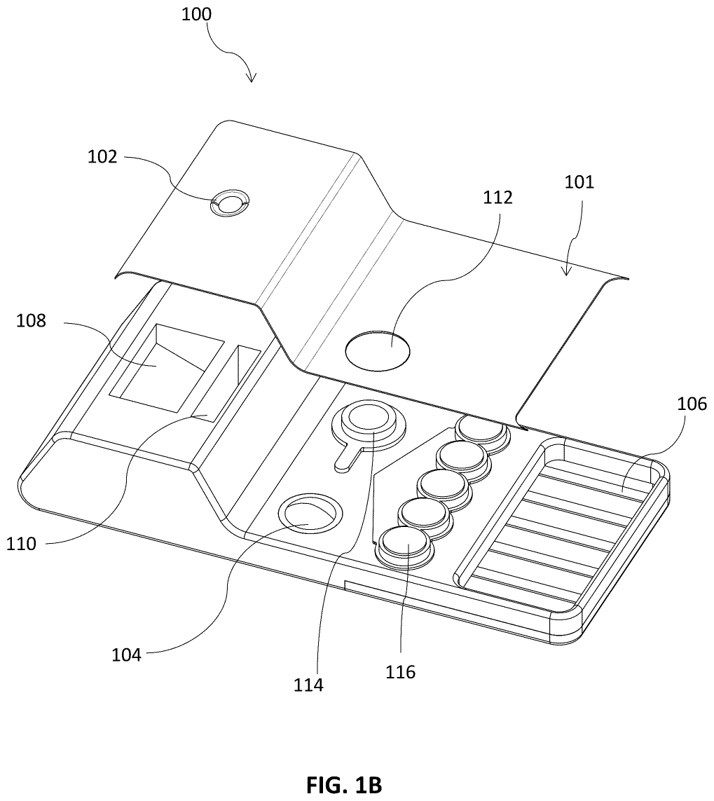

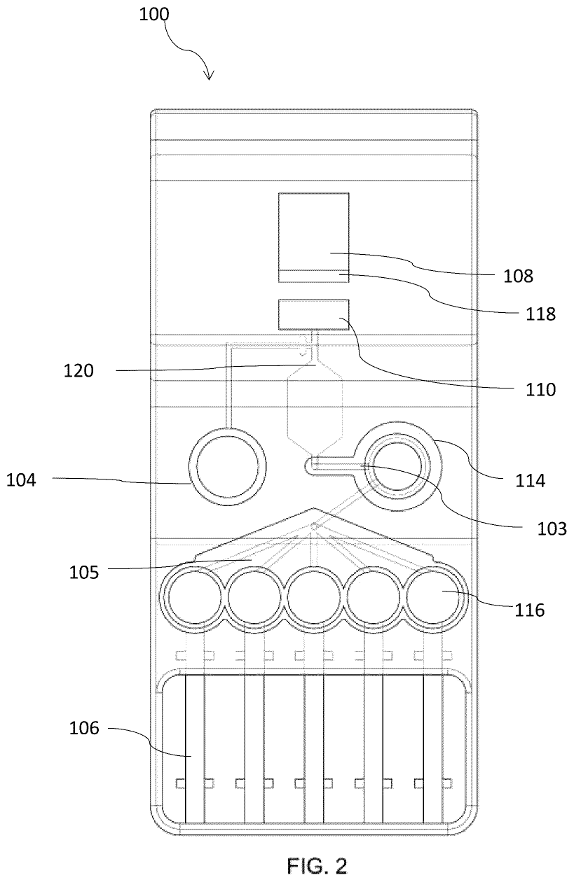

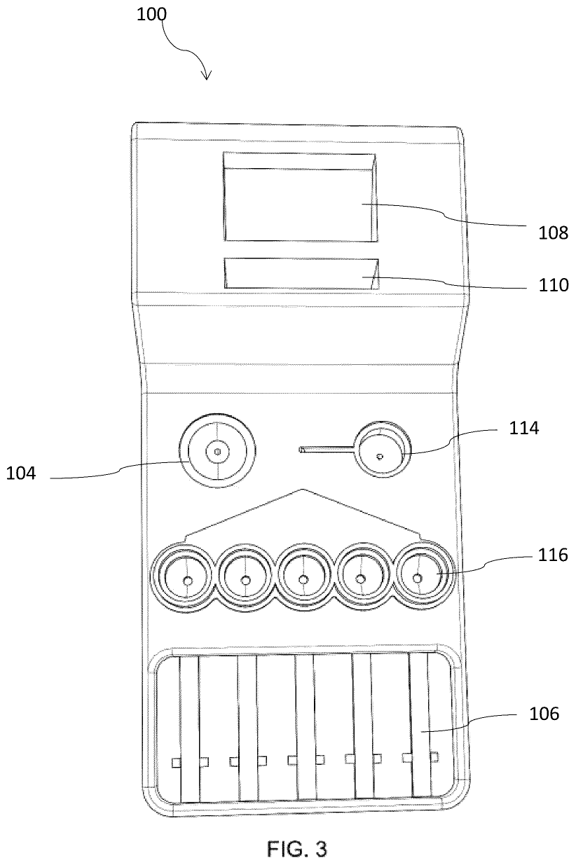

According to the embodiment of the present invention, an automated cartridge for detection of a plurality of analytes in a blood sample of a person is shown in FIG. 1A-FIG. 1B and FIG. 2-FIG. 3. Referring to FIG. 1A, the cartridge 100 comprises a blood tube receiver 102, a pumping mechanism 104 and a plurality of lateral flow strips 106. In reference to FIG. 1B to FIG. 3, the cartridge 100 comprises various components of the cartridges including a blood tube receiver 102, an overflow channel 103, a pumping mechanism 104, a plurality of incubation well channels 105 and a plurality of lateral flow strips 106, a blood collection chamber 108, a plasma separator 118, a plasma chamber 110, a plasma metering chamber 120, a pumping mechanism 104, an overflow chamber 114, a plurality of incubation wells 116 and a plurality of lateral flow strips 106. The cartridge 100 comprises a cartridge cover 101, which is an essential component for maintaining the integrity and functionality of the entire system and provides compactness to the cartridge. The cartridge cover 101 provides a secure, protective seal over the cartridge assembly, ensuring that all internal components, including the blood tube receiver 102, blood collection chamber 108, and other chambers, remain free from contamination and external interference. The cartridge cover is configured with a hole 112 for the pumping mechanism 104. The invention depicts the cartridge 100 that includes the blood tube receiver 102 to accept a blood tube that is holding the person's collected blood sample. The blood tube receiver 102 is inclined to collect the blood sample as it flows in by gravity from the blood tube. The blood then passes from the blood tube receiver 102 to the blood collection chamber 108. The plasma separator 118 is positioned between the blood collection chamber 108 and the plasma chamber 110 for filtering the plasma component from the blood sample. The plasma separator 118 contains a membrane that effectively separates plasma from the other blood components that includes red blood cells, white blood cells, thrombocytes etc. As blood flows through this separator 118, the membrane retains the red blood cells and other cellular components while allowing plasma to pass through. In the embodiment, the plasma separator 118 may comprise an asymmetrical membrane that allows easy and fast separation of blood plasma. In of the embodiment, asymmetrical polysulfone membrane is used which allows plasma separation in less than 2 minutes with more than 80% high quality plasma yield. In the embodiment, blood starts to sediment immediately on entering blood collection chamber 108 and plasma begin to wick through the membrane and gets collected in plasma chamber 110. From there, the plasma flows into the plasma metering chamber 120. This chamber fills with plasma through capillary action, which is the process where the liquid moves into small spaces without the need for external force. The plasma is held in the metering chamber 120 by capillary stops and a pinch valve, which control the flow and containment of the plasma. Further, the cover 101 comprises an opening 131 designed to accommodate a pushing mechanism. The pumping mechanism 104 is used for pumping the plasma from the plasma metering chamber 120 to the overflow chamber 114. The plasma is routed to the overflow chamber 114 through an overflow channel 103. The pumping mechanism 104 utilizes a pump equipped with an aspirate plunger designed with cantilever snaps to avoid accidental activation. To ensure proper flow direction and avoid any backflow, the system is equipped with pinch valves at the inlet and/or outlet of the pumping chamber. These features collectively ensure smooth, controlled movement of the plasma, keeping it from reversing direction and maintaining efficient transfer to the overflow chamber 114. Then the plasma is transferred from the overflow channel 103 to the plurality of incubation wells 116 through a plurality of incubation channels 105. Each of the incubation wells 116 contains a plurality of magnetic beads, a trehalose plug and a chemical reagent. The mixing of the plasma with the chemical reagents takes place through the magnetic agitation produced by the plurality of the magnetic coils that are placed on the thermally conductive printed circuit board. The magnetic field produced allows for the movement of the magnetic beads in anticlockwise or clockwise direction and thus allows for the mixing of the plasma with the chemical reagent efficiently. Following this, the mixture undergoes an incubation process, which lasts between 10 to 30 minutes at a controlled temperature of 37 degree Celsius. A temperature sensor monitors and maintains this temperature, ensuring that the chemical reactions occur under optimal conditions for accurate results. Due to the constant agitation, the trehalose plug placed underneath the incubation well 116 get dissolved/broken and thus the incubated plasma samples are then transferred from the plurality of incubation wells 116 to the plurality of the lateral flow strips 106. The lateral flow strip 106 comprises a test line and a control line. The test line is used for detecting the presence of the plurality of analytes that includes estradiol, progesterone, testosterone, TSH and FSH. The control line ensures the validity of the test. By passing the plasma through these strips, the system can accurately identify and measure the concentrations of the target hormones or analytes.

In one of the embodiments of the present invention, the trehalose plug is made up of celluloses.

In one of the embodiments of the present invention, the plurality of lateral flow strips comprises nitrocellulose.

In one of the embodiments of the present invention, each of the plurality of incubation wells is encased in a conductive plastic enclosure.

In one of the embodiments of the present invention, the cartridge is made up of thermoplastic material.

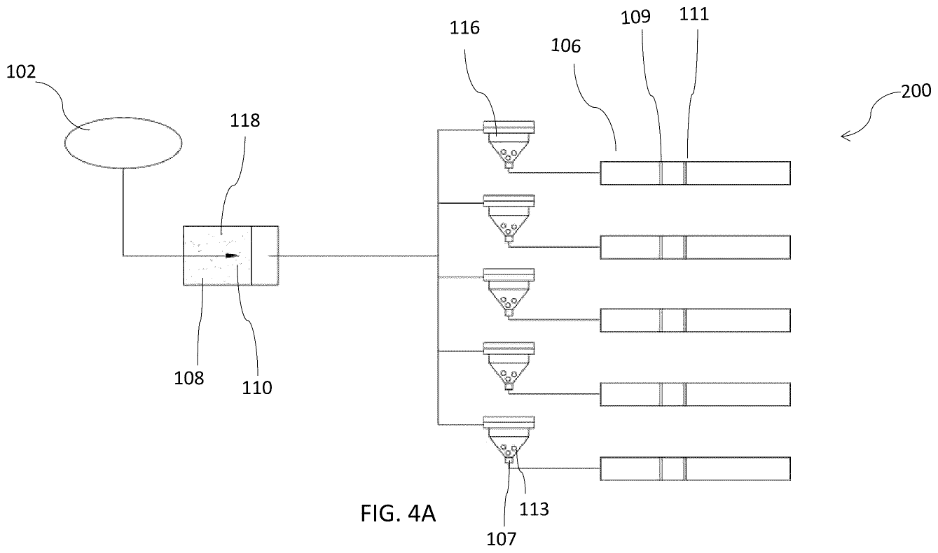

In an embodiment of the present invention, the FIG. 4A illustrates a blood flow process 200 of a blood sample in a cartridge. The process depicts collection of the blood sample from a blood tube receiver 102. The blood samples of a person is collected and stored in a blood collection chamber 108. The plasma separator 118 is positioned between the blood collection chamber 108 and a plasma chamber 110 for filtering the plasma from the blood sample. The plasma separator 118 contains a membrane that effectively separates plasma from the other blood components that includes red blood cells, white blood cells, thrombocytes etc. The membrane retains the blood cells including red cells while allowing plasma to wick into it. By applying a vacuum lyses, the red cells content are passed through the membrane. This combined sedimentation and collection process helps move red cells away from the filter, allowing plasma to effectively reach and pass through the membrane. From there, the plasma flows into the plasma chamber 110. Then the plasma is passed to a plurality of the incubation well 116. Each of the incubation wells 116 contains a plurality of magnetic beads 113, a trehalose plug 107 and a plurality of chemical reagents. The mixing of the plasma with the chemical reagents takes place through the magnetic agitation produced by the plurality of the magnetic coils that are placed on the thermally conductive printed circuit board. Due to the constant agitation, the trehalose plug 107 placed underneath the incubation well 116 gets dissolved/breaks and thus the incubated plasma samples is then transferred from the plurality of incubation wells 116 to a plurality of the lateral flow strips 106. The lateral flow strip 106 comprises a test line 109 and a control line 111. The test line 109 is used for detecting the presence of the plurality of analytes that includes estradiol, progesterone, testosterone, TSH, and FSH in females. The control line 111 ensures the validity of the test. By passing the plasma through these strips, the system can accurately identify and measure the quantity of analytes level present in the person blood sample.

In one of the embodiments of the present invention, referring to FIG. 4B illustrates inter-relationship between plasma separator, pumping mechanism and the plurality of incubation wells of the cartridge. In the embodiment, the cartridge comprises a blood tube receiver. The blood tube receiver is angled to accept a blood tube and collects the blood sample as it flows in by gravity. This blood is then directed to a blood collection chamber 108. In the embodiments, a plasma separator 118 is positioned between the blood collection chamber 108 and the plasma chamber 110 for filtering the plasma component from the blood sample. The separator 118 features a specialized membrane that effectively isolates plasma from other blood components such as red blood cells, white blood cells, and thrombocytes. In the embodiment, the membrane is an asymmetrical polysulfone facilitates rapid plasma separation; often yielding high-quality plasma within 2 minutes. In the embodiment, the pumping mechanism 104 comprises a pump with an aspirate plunger designed with cantilever snaps 144. These snaps 144 are crucial for preventing accidental activation; they securely lock the plunger in place until intentionally engaged. This design enhances both safety and reliability, ensuring that the plunger does not move unintentionally and disrupt the fluid handling process. In addition, the system is carefully managed using one-way valves 146, including cross slit valves. These valves ensure unidirectional fluid flow and prevent backflow, which could otherwise compromise system performance. Additionally, these valves 146 are strategically positioned at the inlet and/or outlet of the pumping chamber, offering precise control over fluid volume and rate. Further, in of the embodiments, the pumping mechanism 104 includes a double stopper mechanism 148 with a dual-seal design. This mechanism precisely controls fluid release and prevents leakage or mixing of reagents. In addition, in one of the embodiments, the cartridge 100 comprises a ferrous mixing element 150 that includes a plurality of magnetic beads 113. In one embodiment, the ferrous mixing element 150 is moved magnetically within the fluid to ensure thorough and uniform mixing. Additionally, in one the embodiments, the cartridge 100 incorporate a foil layer 152 with an air channel. This feature regulates air pressure and manages gas movement, which is crucial for maintaining the correct pressure conditions within the system. Moreover, in one of the embodiments, the separated plasma flows to an overflow chamber 114 and then flows towards a plurality of incubation wells 116. Each of the incubation wells 116 contains a plurality of magnetic beads, a trehalose plug 107 and a chemical reagent 154. The mixing of the plasma with the dry chemical reagents 154 takes place through the magnetic agitation produced by the plurality of the magnetic coils that are placed on the thermally conductive printed circuit board beneath the incubation wells 116. Further, a plurality of lateral flow strip 106 comprises a test line 109 and a control line 111 for detecting the presence of the plurality of analytes that includes estradiol, progesterone, testosterone, TSH and FSH.

In one of the embodiments of the present invention, the plurality of magnetic beads are ceramic magnetic beads and measuring 1.5 mm×4 mm.

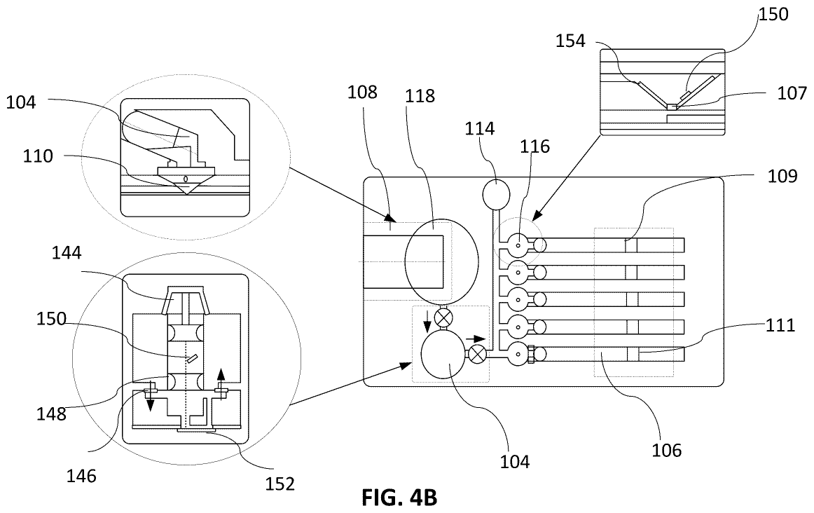

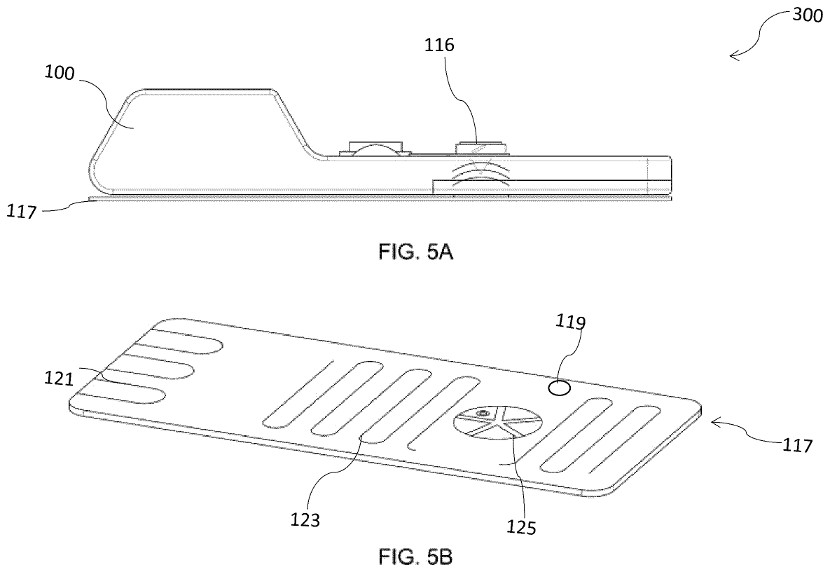

In an embodiment of the present invention, the invention 300 as shown in FIG. 5A depicts coupling of a cartridge 100 with a thermally conductive printed circuit board 117. The invention discloses a thermally conductive printed circuit board 117 attached beneath a plurality of incubation wells 116. Each of the incubation wells 116 contains a plurality of magnetic beads, a trehalose plug with a cellulose membrane and a chemical reagent. The mixing of the plasma with the chemical reagents takes place through the magnetic agitation produced by the plurality of the magnetic coils that are placed on the thermally conductive printed circuit board 117. The magnetic field produced allows for the movement of the magnetic beads in anticlockwise or clockwise direction and thus allows for the mixing of the plasma with the chemical reagent efficiently. Following this, the mixture undergoes an incubation process between 10 to 30 minutes at a controlled temperature of 37 degree Celsius. A temperature sensor then monitors and maintains this temperature, ensuring that the chemical reactions occur under optimal conditions for accurate results. Due to the constant agitation, the trehalose plug placed underneath the incubation well 116 get dissolved and thus the incubated plasma samples are transferred from the plurality of incubation wells 116 to the plurality of the lateral flow strips. The lateral flow strips comprise a test line and a control line that is used for detecting the presence of the plurality of analytes that includes estradiol, progesterone, testosterone, TSH, and FSH.

In an embodiment of the present invention, the FIG. 5B depicts the overview of the overall components of the thermally conductive printed circuit board 117. The thermally conductive printed circuit board 117 can be used separately or in combination with the cartridge. The thermally conductive printed circuit board (PCB) 117 comprises a power source input 121 for providing electricity to the PCB 117, a plurality of printed heater 123 for providing heat within the PCB 117, a sensor 119 for measuring and maintaining the desired temperature for carrying out the incubation process efficiently and a plurality of the magnetic coil 125 for creating a magnetic field to rotate the magnetic beads in anticlockwise or clockwise direction. In the embodiment, the power source input 121 may be a dry cell or rechargeable battery or Li-ion battery.

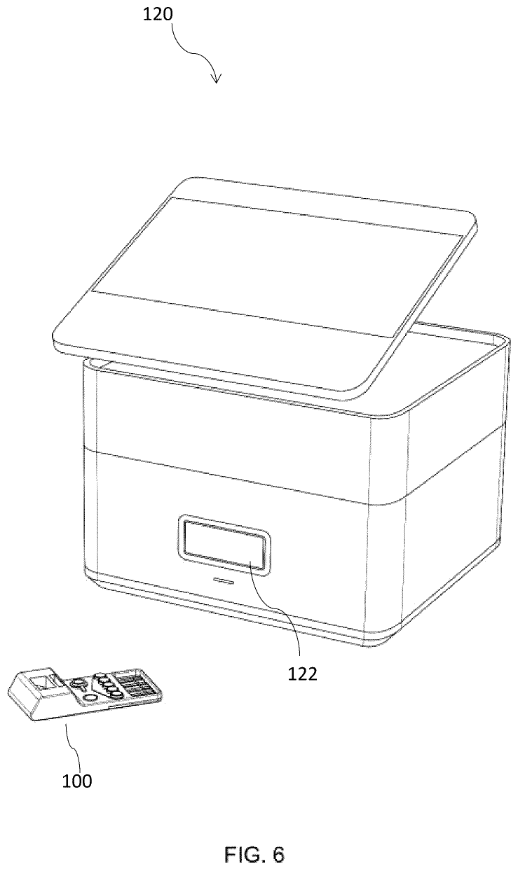

In an embodiment of the present invention, the FIG. 6 depicts a system comprising a cartridge 100 and a measuring instrument 120. The system discloses a measuring instrument 120 that comprises a cartridge receiver 122 having a motor to facilitate the entry of the cartridge 100 into the system. The cartridge 100 is configured to have the person's blood sample. The cartridge is then inserted inside the cartridge receiver 122 of the instrument 120. The person can then start the test run by providing the inputs through a touchscreen interface module. The instrument 120 then performs all the tasks necessary to run the assay. The instrument 120 then displays the results and sends the data to a remote server. The person can then remove the used cartridge 100 and dispose of. Further, the instrument 120 is ready to run another test using a new sample from a new cartridge 100.

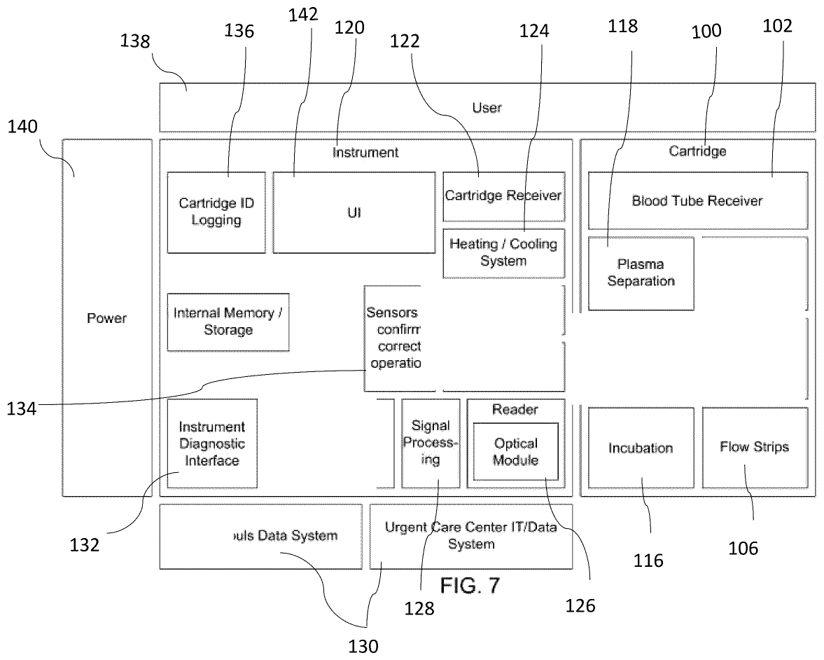

In an embodiment of the present invention, the FIG. 7 depicts a system 400 for detection of the presence of a plurality of analytes in a blood sample. The system comprises a measuring instrument 120, a cartridge 100, a data system 130 and a power source 140 and a user 138. The measuring instrument 120 further comprises a cartridge ID logging module 136 to store the user 138 credentials, a temperature control module 124 to maintain temperature conditions for an effective incubation, a touchscreen interface module 142 for displaying the results of a tested blood sample of a user 138, a diagnostic module 132 for scheduling system maintenance that includes software updates, firmware updates and a signal processing module 128 for calculating results by analysing a fluorescence signal for a plurality of lateral flow strips within a inserted cartridge 100. The system includes a power source 140 to power the component or modules involved in the system. Further, the system includes a sensor module 134 to ensure that the instrument and its modules are functioning correctly, an imaging module integrated with an optical module 126 for processing image data, detecting test lines, reading results, and correcting artifacts. The system further includes cartridge 100 that comprises a blood tube receiver 102, a plasma separator 118, an overflow chamber, a plurality of incubation wells 116 and a plurality of lateral flow strips 106. The invention depicts a cartridge 100 that includes a blood tube receiver 102 to accept a blood tube having patient's collected blood sample. The plasma separator 118 is used for filtering the plasma component from the blood sample. The plasma separator 118 contains a membrane that effectively separates plasma from the other blood components that includes red blood cells, white blood cells, thrombocytes etc. As blood flows through this separator 118, the membrane retains the red blood cells and other cellular components while allowing plasma to pass through. The overflow chamber is used for receiving the plasma from a plasma metering chamber though a pumping mechanism. Each of the incubation wells 116 contains a plurality of magnetic beads, a trehalose plugs and a chemical reagent. The mixing of the plasma with the chemical reagents takes place through the magnetic agitation produced by the plurality of the magnetic coils that are placed on the thermally conductive printed circuit board. The magnetic field produced allows for the movement of the magnetic beads in anticlockwise or clockwise direction and thus allows for the mixing of the plasma with the chemical reagent efficiently. Further, the lateral flow strip 106 comprises a test line and a control line for detecting the presence of the plurality of analytes that includes estradiol, progesterone, testosterone, TSH and FSH. By passing the plasma through these strips, the system can accurately identify and measure the concentrations of the target hormones or analytes. The system further includes a data system 130 equipped with a data management component that stores all relevant information, including the raw instrument data and calculated test results. This data is uploaded to a cloud-based system, thus allowing for easy access to test results from anywhere, facilitates data management, and ensures compliance with privacy regulations. The cloud-based approach also enables the integration of results with broader data systems and supports further analysis or sharing with healthcare professionals.

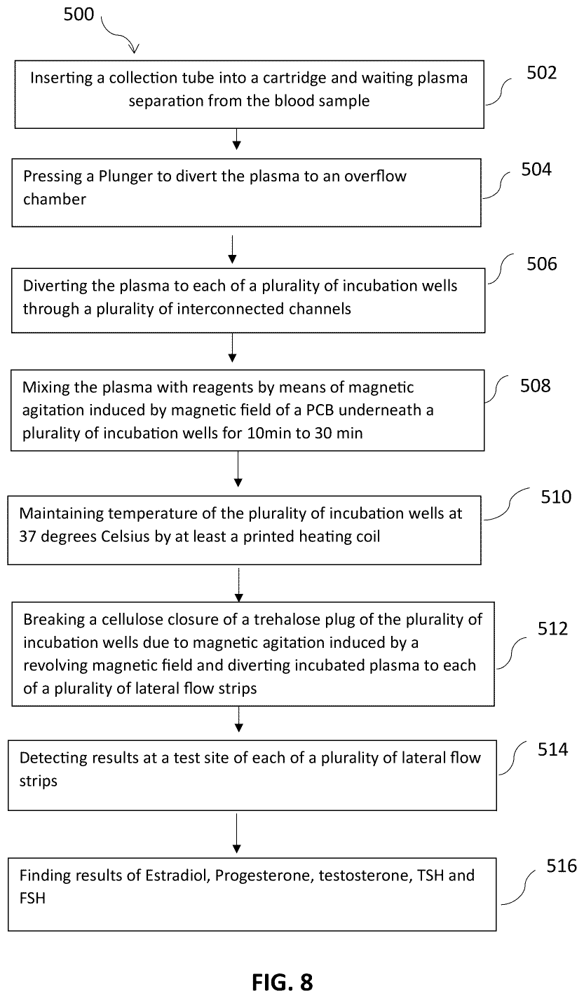

In an embodiment of the present invention, the invention as shown in FIG. 8 discloses a fully automated method 500 of detecting a plurality of analytes in a blood sample comprising steps of inserting a collection tube into a cartridge and waiting plasma separation from the blood sample 502, pressing a Plunger to divert the plasma to an overflow chamber 504, diverting the plasma to each of a plurality of incubation wells through a plurality of interconnected channels 506, mixing the plasma with reagents by means of magnetic agitation induced by magnetic field of a PCB underneath a plurality of incubation wells for 10 min to 30 min 508, maintaining temperature of the plurality of incubation wells at 37 degrees Celsius by at least a printed heating coil 510, breaking a cellulose closure of a trehalose plug of the plurality of incubation wells due to magnetic agitation induced by a revolving magnetic field and diverting incubated plasma to each of a plurality of lateral flow strips 512, and detecting results at a test site of each of a plurality of lateral flow strips; and finding results of the plurality of analytes 514. In the embodiment, the method the plurality of analytes includes estradiol, progesterone, testosterone, TSH and FSH.

In one of the embodiments, the test line detects the plurality of analytes and the control line ensures the validity of the test. By passing the plasma through the lateral flow strips, the system can accurately identify and measure the concentrations of the target hormones or analytes.

It should be understood that the examples provided herein are intended only for purposes of illustration and any number of other implementations is also contemplated. Additionally, the referenced examples (including the described rules and/or other techniques) can be combined in any number of ways.

Although an overview of the inventive subject matter has been described with reference to specific example implementations, various modifications and changes can be made to those implementations without departing from the broader scopes of implementation of the present disclosure. Such implementation of the inventive subject matter can be referred to herein, individually or collectively, by the term “invention” merely for convenience without intending to voluntarily limit the scope of this application to any single disclosure or inventive concept if more than one is, in fact is disclosed.

The implementations illustrated herein are described in sufficient detail to enable those skilled in the art to practice the teachings disclosed. Other implementations can be used and derived therefrom, such that structural substitutions and changes can be made without departing from the scope of this disclosure. The Detailed Description, therefore, is not to be taken in a limiting sense, and the scope of various implementations is defined only by the appended claims, along with the full range of equivalents to which such claims are entitled.

As used herein, the term “or” can be construed in either an inclusive or exclusive sense. Moreover, plural instances can be provided for resources or structures described herein as a single instance. These and other variations, modifications, additions, and improvements fall within a scope of implementations of the present disclosure as represented by the appended claims. The specification and drawings are, accordingly, to be regarded in an illustrative rather than a restrictive sense.

What is claimed is:

1. An automated cartridge for detection of a plurality of analytes in a blood sample of a person, comprising:
a blood tube receiver to receive the blood sample into the automated cartridge;
a plasma separator positioned between a blood collection chamber and a plasma chamber, wherein the plasma separator filters plasma from the blood sample into the plasma chamber;
a plasma metering chamber receiving said plasma, wherein a pumping mechanism pumps the plasma to flow from the plasma metering chamber to an overflow chamber;
wherein the overflow chamber diverts the plasma to a plurality of incubation wells; wherein the plurality of incubation wells comprises a plurality of magnetic beads and reagents;
wherein a plurality of lateral flow strips configured to receive the incubated plasma from the plurality of incubation wells after a breakable barrier is broken due to magnetic agitation under a magnetic field.

2. The automated cartridge as claimed in claim 1, wherein the pumping mechanism comprises a pump with an aspirate plunger having cantilever snaps or a pump with a double stopper.

3. The automated cartridge as claimed in claim 1, wherein the pumping mechanism comprises a valve to prevent backflow of the sample.

4. The automated cartridge as claimed in claim 1, further comprises a thermally conductive printed circuit board comprising:
a plurality of magnetic coils configured to generate a revolving magnetic field;
a plurality of printed heaters;
a temperature sensor; and
a power input source.

5. (canceled)

6. (canceled)

7. The automated cartridge as claimed in claim 4, wherein the plurality of magnetic beads are moved under a magnetic field induced by means of said thermally conductive printed circuit board underneath the plurality of incubation wells allowing the mixing of the blood sample with the chemical reagents.

8. The automated cartridge as claimed in claim 1, wherein the plasma is incubated for 10-30 min at 37 degree Celsius in the plurality of incubation wells.

9. The automated cartridge as claimed in claim 1, wherein the plurality of magnetic beads are ceramic magnetic beads.

10. The automated cartridge as claimed in claim 1, wherein the breakable barrier is a trehalose plug made up of cellulose.

11. The automated cartridge as claimed in claim 1, wherein the plurality of lateral flow strips comprises a control line and a test line to detect the presence of the plurality of analytes.

12. The automated cartridge as claimed in claim 1, wherein the presence of the plurality of analytes is selected from a group comprising:
estradiol, progesterone, testosterone, TSH, and FSH.

13. The automated cartridge as claimed in claim 1, wherein the plurality of lateral flow strips comprises nitrocellulose.

14. The automated cartridge as claimed in claim 1, wherein each of the plurality of incubation wells is surrounded by a conductive plastic.

15. The automated cartridge as claimed in claim 1, wherein the cartridge is made up of thermoplastic material.

16. A computer-implemented system for detection of a plurality of analytes in a blood sample, comprising:
a measuring instrument powered by a power source, wherein the measuring instrument comprises:
a cartridge ID logging module to store a person's credentials;
a touchscreen interface module configured for displaying results of a tested blood sample of a person;
a signal processing module that calculates results by analyzing a fluorescence signal for a plurality of lateral flow strips within an insertable cartridge;
an imaging module integrated with an optical module for processing image data, detecting test lines, reading results, and correcting artifacts;
a cartridge receiver having an internal motor that engages and pulls the insertable cartridge inward, wherein the insertable cartridge, hereinafter referred to as cartridge, comprises:
a blood tube receiver to receive the blood sample into the cartridge;
a plasma separator positioned between a blood collection chamber and a plasma chamber, said plasma separator filters plasma from the blood sample into the plasma chamber;
a plasma metering chamber that receives the plasma wherein a pumping mechanism pumps the plasma to flow from the plasma metering chamber to an overflow chamber;
wherein the overflow chamber diverts the plasma to a plurality of incubation chambers; wherein the plurality of incubation chambers includes a plurality of magnetic beads and reagents;
wherein a plurality of lateral flow strips configured to receive the incubated plasma from the plurality of incubation chambers after a breakable barrier is broken due to magnetic agitation under a magnetic field; and
a data system for storing usage data and measured result data of the person virtually.

17. The computer-implemented system for detection as claimed in claim 16, wherein the data system is in communication with a remote server to store data virtually.

18. (canceled)

19. The computer-implemented system for detection as claimed in claim 16, further comprising a thermally conductive printed circuit board including a plurality of magnetic coils, a plurality of printed heaters, a temperature sensor and a power input source, wherein the thermally conductive printed circuit board is configured to generate a revolving magnetic field in each of the plurality of magnetic coils.

20. A method of detecting a plurality of analytes in a blood sample utilizing a measuring instrument, the method comprising steps of:
inserting a collection tube into a cartridge and waiting for plasma separation from the blood sample;
then pressing a plunger to divert the plasma to an overflow chamber;
then diverting the plasma to each of a plurality of incubation wells through a plurality of interconnected channels;
then mixing the plasma with reagents by means of magnetic agitation induced by magnetic field from a PCB underneath a plurality of incubation wells;
then maintaining the plurality of incubation wells at a predetermined temperature;
then breaking a breakable barrier of the plurality of incubation wells due to magnetic agitation induced by a revolving magnetic field and diverting incubated plasma to each of a plurality of lateral flow strips;
then detecting results at a test site of each of the plurality of lateral flow strips; and finding results of the plurality of analytes.

21. The method of claim 20, wherein the plasma is incubated for 10 to 30 minutes at 37 degree Celsius in the plurality of incubation wells.
